(19) United States
(12) Patent Application Publication
(10) Pub. No.: US 2017/0209373 A1
(43) Pub. Date: Jul. 27, 2017

(54) MULTIFUNCTIONAL POLYMERIC NANOPARTICLE FOR DIAGNOSIS OR TREATMENT OF CEREBRAL DISEASES AND A PREPARATION METHOD THEREOF

(71) Applicant: KOREA INSTITUTE OF SCIENCE AND TECHNOLOGY, Seoul (KR)

(72) Inventors: Sehoon KIM, Seoul (KR); Youngsoo KIM, Seoul (KR); Youngsun KIM, Seoul (KR); Keunsoo JEONG, Seoul (KR); Ajay SINGH, Buffalo, NY (US)

(21) Appl. No.: 15/361,996

(22) Filed: Nov. 28, 2016

(30) Foreign Application Priority Data
Jan. 27, 2016 (KR) 10-2016-0115881

(51) Int. Cl. A61K 9/16; A61K 31/12; A61K 49/00; A61K 9/00

(52) U.S. Cl. CPC A61K 9/1641; A61K 9/1694; A61K 9/0019; A61K 31/12; A61K 49/0054

(57) ABSTRACT

The present invention relates to a drug delivery system (DDS) for crossing the blood-brain barrier (BBB) formed by self-assembly of an amphiphilic block copolymer, comprising a self-assembled structure having an average diameter of 5 nm to 20 nm of a core-shell structure comprising a hydrophobic core and a hydrophilic shell; and a hydrophobic drug supported in the hydrophobic core of the self-assembled structure, a pharmaceutical composition for preventing or treating cerebral diseases comprising the drug delivery system as an active ingredient, and a preparation method of the drug delivery system.

### CROSS REFERENCE TO RELATED PATENT APPLICATIONS

This application claims the benefit under 35 U.S.C. §119, the priority of Korean Patent Application No. 10-2016-0115881, filed Jan, 27, 2016,in the Korean Intellectual Property Office, the disclosures of which are hereby incorporated by the references.

### TECHNICAL FIELD

The present invention relates to a drug delivery system (DDS), which crosses the blood-brain barrier (BBB) formed by self-assembly of an amphiphilic block copolymer, comprising a self-assembled structure having an average diameter of 5 nm to 20 nm of a core-shell structure with a hydrophobic core and a hydrophilic shell; and a hydrophobic drug supported in the hydrophobic core of the self-assembled structure, a pharmaceutical composition for preventing or treating cerebral diseases comprising the drug delivery system as an active ingredient, and a method for preparing the drug delivery system.

### BACKGROUND ART

The blood-brain barrier (BBB) is a cellular barrier composed of tight junctions having a high magnitude of electrical resistance of at least 0.1 Ωm between vascular endothelial cells that are adjacent to related pericytes and astrocytes, and it acts as a barrier having highly selective permeability to separate circulating blood from brain extracellular fluids in the central nervous system (CNS), thereby playing a role of a gateway to protect the central nervous system by regulating the entry and exit of nutrients and other substances to the brain. Normally, the BBB not only selectively transfers molecules such as glucose and amino acids that are essential for brain function, but also passes water, some gases, and fat-soluble molecules by passive diffusion. On the other hand, the BBB blocks the entry and exit of lipophilic, potential neurotoxins by an active transport mechanism mediated by permeable p-glycoprotein. The BBB is formed along all capillaries and is composed of tight junctions around capillaries that do not exist in normal circulation. As such, although the BBB serves to prevent the transport of bacteria, pathogens, and potential hazardous substances in blood to the brain that can be carried through the blood, due to the barriers of such blood vessels, most drugs for the central nervous system show low efficiency of transcranial delivery, and therefore, in order to compensate for this, such drugs are administered at a high dose, which may cause serious side effects in surrounding organs.

Therefore, there is a need to find an efficient drug delivery system that can cross the BBB in order to ensure the therapeutic effects of chemodrugs, at the same time preventing negative systemic effects.

### DISCLOSURE

### Technical Problem

Using the self-assembling properties of amphiphilic block copolymers, the present inventors undertook diligent research efforts in order to discover a drug delivery system that can cross the BBB and deliver a drug specifically to the brain even when the drug is administered systematically, and as a result, the present invention has been completed by confirming that, a drug delivery system, which comprises a self-assembled structure having an average diameter of 5 nm to 20 nm of a core-shell structure with a hydrophobic core and a hydrophilic shell, formed by self-assembly of an amphiphilic block copolymer by mixing an amphiphilic block copolymer comprising a hydrophilic block of poly(ethylene oxide) (PEO) and a hydrophobic block of poly(propylene oxide) (PPO); and a hydrophobic drug at a predetermined ratio, in which the hydrophobic drug to be delivered is supported in the hydrophobic core, can deliver the hydrophobic drug specifically to the brain and release the drug, even when systematically administered.

### Technical Solution

The first aspect of the present invention provides a drug delivery system (DDS), which crosses the blood-brain barrier (BBB), comprising: a self-assembled structure having an average diameter of 5 nm to 20 nm of a core-shell structure with a hydrophobic core and a hydrophilic shell, in which a hydrophobic block of an amphiphilic block copolymer is positioned inside the structure and a hydrophilic block of the amphiphilic block copolymer is positioned out of the structure, respectively, and the amphiphilic block copolymer comprises the hydrophilic block of poly(ethylene oxide) (PEO) and the hydrophobic block of poly(propylene oxide) (PPO); and a hydrophobic drug supported in the hydrophobic core of the self-assembled structure, wherein the ratio of the amphiphilic block copolymer to the hydrophobic drug is adjusted for crossing the blood-brain barrier.

The second aspect of the present invention provides a pharmaceutical composition for preventing or treating cerebral diseases, comprising the drug delivery system according to the first aspect of the present invention as an active ingredient.

The third aspect of the present invention provides a method for preparing a drug delivery system, which crosses the blood-brain barrier, having an average diameter of 5 nm to 20 nm of a core-shell structure with a hydrophilic shell, a hydrophobic core and a hydrophobic drug supported therein, comprising: a first step of airblowing a solution containing a hydrophobic drug, and a amphiphilic block copolymer comprising a hydrophilic block of poly(ethylene oxide) (PEO) and a hydrophobic block of poly(propylene oxide) (PPO) in an organic solvent, in which the ratio of the amphiphilic block copolymer to the hydrophobic drug is adjusted for crossing the blood-brain barrier, to evaporate the organic solvent; and a second step of adding a water-containing solvent to the product obtained from the previous step followed by dispersion by an ultrasonic disperser.

Hereinbelow, the present invention will be described in more detail.

The present invention is based on a discovery that when a drug delivery system, a self-assembled structure of a core-shell structure with a hydrophobic core and a hydrophilic shell formed by self-assembly, in which a hydrophobic block of an amphiphilic block copolymer is positioned inside the structure and a hydrophilic block of the amphiphilic block copolymer is positioned out of the structure, respectively, in an aqueous solvent and the amphiphilic block copolymer comprises the hydrophilic block of poly(ethylene oxide) (PEO) and the hydrophobic block of poly(propylene oxide) (PPO), is prepared by supporting a hydrophobic drug in its hydrophobic core, it is mainly accumulated in the brain by crossing the blood-brain barrier even when it is systemically administered by intravenous injection if the size of the drug delivery system is adjusted to have an average diameter of 5 nm to 20 nm. Specifically, by adjusting the ratio of the amphiphilic block copolymer to the internally supported hydrophobic drug, the present inventors showed that it is possible to provide a drug delivery system having the above-mentioned size and/or effect.

The present invention can provide a drug delivery system (DDS), which crosses the blood-brain barrier (BBB), comprising: a self-assembled structure having an average diameter of 5 nm to 20 nm of a core-shell structure with a hydrophobic core and a hydrophilic shell, in which a hydrophobic block of an amphiphilic block copolymer is positioned inside the structure and a hydrophilic block of the amphiphilic block copolymer is positioned out of the structure, respectively, and the amphiphilic block copolymer comprises the hydrophilic block of poly(ethylene oxide) (PEO) and the hydrophobic block of poly(propylene oxide) (PPO); and a hydrophobic drug supported in the hydrophobic core of the self-assembled structure.

For example, the drug delivery system may comprise the drug delivery system comprises the hydrophobic drug and the amphiphilic block copolymer at a mass ratio (w/w) of (0.025 to 2):20. Alternatively, the drug delivery system may comprise the hydrophobic drug and the amphiphilic block copolymer at a mass ratio (w/w) of (0.05 to 1):20 or (0.1 to 0.5):20. If the ratio of the hydrophobic drug to the amphiphilic block copolymer is less than 0.025 mg/20 mg, the drug supporting efficiency may be low, and if the ratio is over 5 mg/20 mg, as the size of the formed drug delivery system becomes larger, it is difficult to cross the blood-brain barrier, and the delivery efficiency to the brain may be lowered.

For example, the amphiphilic block copolymer is a triblock copolymer having an average molecular weight of 1,500 Da to 20,000 Da. Alternatively, it may be a triblock copolymer having an average molecular weight of 8,000 Da to 15,000 Da, but is not limited thereto. For example, the amphiphilic block copolymer may be a block copolymer comprising PEO and PPO blocks at a ratio of (2.5 to 6):1 based on the numbers of EO and PO units constituting the hydrophilic PEO block and hydrophobic PPO block, respectively. The amphiphilic block copolymer can be synthesized and used so as to comprise the hydrophilic and hydrophobic blocks at the above-mentioned ratio and to have a molecular weight in the above-mentioned range, or commercially available products such as Pluronic F-127 or F-68 corresponding thereto can be purchased and used.

For example, the hydrophobic drug is an anticancer agent, antioxidant agent, anti-inflammatory agent, contrast agent, or combination thereof, but is not limited thereto. As the anticancer, antioxidant, and anti-inflammatory agents, a drug capable of being supported in a hydrophobic core of a particle formed by an amphiphilic polymer can be used as the hydrophobic drug without limitation.

Non-limiting examples of the anticancer agent may include curcumin, Doxorubicin (Dox), Paclitaxel (Taxol), and Cisplatin.

Non-limiting examples of the antioxidant agent may include vitamin E, alpha-carotene, and anthocyanine.

Non-limiting examples of the anti-inflammatory agent may include Methotrexate (MTX), Leflunomide, and Tacrolimus.

Meanwhile, a contrast agent is a substance that functions to provide clear images so that a disease occurring at a site which cannot be confirmed by simple imaging can be diagnosed early and treated, and contrast agents for magnetic resonance imaging (MRI), contrast agents for computed tomography (CT), contrast agents for positron emission tomography (PET), ultrasound contrast agents, and fluorescent contrast agents, etc., may be used. Non-limiting examples of the contrast agent may include contrast agents for magnetic resonance imaging (MRI), which are paramagnetic or superparamagnetic substances of a transition metal ion including gadolinium (Gd), manganese (Mn), copper (Cu), and chromium (Cr), of hydrophobic complexes of the transition metal ion including gadopentetate dimeglumine (Gd-DTPA) and gadoterate meglumine (Gd-DOTA), of fluorine-containing compounds including perfluorocarbons and perfluoropropane, of iron oxide-based, manganese-based, copper-based, and chromium-based nanoparticles, and compounds obtained by modifying the surface of the nanoparticles with a hydrophobic substance, etc.; contrast agents for computed tomography (CT) of iodinated hydrophobic substances derived from iodinated poppy seed oil, and nanoparticles composed of a metal element including bismuth (Bi), gold (Au), and silver (Ag), etc.; contrast agents for positron emission tomography (PET) of a radioactive isotope including ^{99m}Tc, ¹²³I, ¹⁶⁶Ho, ¹¹¹In, ⁹⁰Y, ¹⁵³Sm, ¹⁸⁶Re, ¹⁸⁸Re, ⁶⁸Ga, and ¹⁷⁷Lu, and of hydrophobic complexes of the radioactive isotope prepared using diethylenetriaminepentaacetate (DTPA), etc.; ultrasonic contrast agents which are hydrophobic compounds of perfluoropropane, perfluorohexane, sulfur hexafluoride, perfluoropentane, and decafluorobutane, etc.; and fluorescent contrast agents such as fluorescein, rhodamine, Nile red, Cy-3, and Cy-5, etc., and near-infrared fluorescent substances described below can be included without limitation.

For example, the drug delivery system may further comprise a near-infrared fluorescent substance in the hydrophobic core of the self-assembled structure. In particular, the near-infrared fluorescent substance may be comprised at a mass ratio (w/w) of (0.025 to 0.5):20, (0.025 to 0.3):20, or (0.05 to 0.25):20 relative to the amphiphilic block copolymer, wherein the sum of the masses of the hydrophobic drug and the near-infrared fluorescent substance may be adjusted to have a mass ratio (w/w) of (0.05 to 2.5):20, (0.1 to 1):20, or (0.1 to 0.3):20 relative to the mass of the amphiphilic block copolymer. For example, the near-infrared fluorescent substance may be selected from fluorescent substances exhibiting fluorescence in a range of 640 nm to 1,000 nm. For example, it may be a series of indane derivatives or a series of 1,3-indandione-based derivatives comprising a carbazole ring or aniline ring, but is not limited thereto. Specifically, non-limiting examples of the near-infrared fluorescent substance may include 2,2′-(2-((9-ethyl-9H-carbazol-3-yl)methylene)-1H-indene-1,3(2H)-diylidene)dimalononitrile, (Z)-2-(2-((9-ethyl-9H-carbazol-3-yl)methylene)-3-oxo-2,3-dihydro-1H-inden-1-ylidene)malononitrile, (Z)-2-(2-(4-(dimethylamino)benzylidene)-3-oxo-2,3-dihydro-1H-inden-1-ylidene)malononitrile, 2,2′-(2-(4-(dimethylamino)benzylidene)-1H-indene-1,3(2H)-diylidene)dimalononitrile, 2,2′-(1H-indene-1,3(2H)-diylidene)dimalononitrile, (2,2′-(1H-indene-1,3(2H)-diylidene)dimalonic acid, (2E,2′Z)-diethyl 2,2′-(1H-indene-1,3(2H)-diylidene)bis(2-cyanoacetate), (2E,2′Z)-2,2′-(1H-indene-1,3(2H) -diylidene)bis(2-cyanoacetic acid), (E)-ethyl 2-cyano-2-(3-(dicyanomethylene)-2,3-dihydro-1H-inden-1-ylidene)acetate, 2,2′,2″-([1,2′-biindenylidene]-1′,3,3′(2H)-triylidene)trimalonic acid, 2,2′,2″-((1Z,1′Z,3E,3′E)-[1,2′-biindenylidene]-1′,3,3′(2H)-triylidene)tris(2-cyanoacetic acid), triethyl 2,2′,2″-((1Z, 1′Z,3E,3′E)-[1,2′-biindenylidene]-1′,3,3′(2H)-triylidene)tris(2-cyanoacetate), and 2,2′,2″-([1,2′-biindenylidene]-1′,3,3′(2H)-triylidene)trimalononitrile.

The drug delivery system of the present invention can be used as a pharmaceutical composition for preventing or treating cerebral diseases comprising the drug delivery system as an active ingredient. For example, the pharmaceutical composition may further comprise a carrier and may be provided in an injectable form, but is not limited thereto.

In a specific exemplary embodiment of the present invention, it was confirmed that even when the drug delivery system comprising curcumin as a drug was intravenously injected in the form of an aqueous dispersion, it was efficiently delivered to the brain by crossing the blood-brain barrier, and furthermore, in the brain tumor model, as it was selectively delivered to the tumor site of the brain and released the drug, it was confirmed that the drug delivery system suppressed the growth of tumor and exhibited a therapeutic effect of making the tumor size significantly smaller, even when used in an amount as low as 1/2,000 relative to a simple solution of the same drug.

Further, the drug delivery system of the present invention can be prepared via a first step of airblowing a solution containing a hydrophobic drug, and a amphiphilic block copolymer comprising a hydrophilic block of poly(ethylene oxide) (PEO) and a hydrophobic block of poly(propylene oxide) (PPO) in an organic solvent, to evaporate the organic solvent; and a second step of adding a water-containing solvent to the product obtained from the previous step followed by dispersion by an ultrasonic disperser.

For example, in the second step, the water-containing solvent may be used in which the final concentration of the amphiphilic block copolymer used in the solution is set to be in a range of 5 mg/mL to 50 mg/mL. More specifically, the concentration of the amphiphilic block copolymer used in the solution may be in a range of 10 mg/mL to 30 mg/mL, but is not limited thereto.

For example, as the organic solvent, a volatile organic solvent capable of simultaneously dissolving a hydrophobic drug and a polymeric surfactant, that is, an amphiphilic polymer, can be used without limitation. Specifically, the organic solvent may be dichloromethane, tetrahydrofuran, chloroform, or a mixture thereof, but is not limited thereto.

For example, the solution of the first step may further comprise a near-infrared fluorescent substance, but is not limited thereto, and the content thereof is as described above. For example, the near-infrared fluorescent substance is preferably a fluorescent substance which does not cause self-quenching of fluorescence, even if it is densely concentrated.

For example, if the hydrophobic drug is a molecule which exhibits fluorescence, the near-infrared fluorescent substance may be selected to have an absorption spectrum in the fluorescence wavelength range of the hydrophobic drug. When combined as above, in the case of exciting at the absorption wavelength of the hydrophobic drug, by a phenomenon of fluorescence energy transfer, fluorescence generated therefrom is absorbed by adjacent near-infrared fluorescent substances supported therewith, thereby generating fluorescence thereof, and by detecting the fluorescence of the fluorescent substances, it can be confirmed whether the drug delivery system was delivered to a lesion by monitoring the position thereof, and at the same time, it can be confirmed from changes in the intensity thereof whether the drug was released.

### Advantageous Effects

Since the drug delivery system of the present invention i) is formed by self-assembly of an amphiphilic block copolymer comprising a hydrophilic block of poly(ethylene oxide) (PEO) and a hydrophobic block of poly(propylene oxide) (PPO), ii) has a core-shell structure with a hydrophobic core and a hydrophilic shell so that it is capable of supporting a hydrophobic drug in the hydrophobic core thereof, and iii) has a small size of an average diameter of 5 nm to 20 nm, even when administered systematically by intravenous injection, it does not become accumulated in other organs such as liver, pancreas, and kidney, and is able to cross the blood-brain barrier and be efficiently delivered to the brain, enabling to release the drug specifically to the lesion, and therefore the drug delivery system can be valuably used for preventing or treating cerebral diseases by supporting various drugs inside.

### DESCRIPTION OF THE DRAWINGS

FIG. 1 shows the chemical structures of a drug and a fluorescent dye used in nanoparticles containing the hydrophobic drug and/or the fluorescent dye and a schematic design diagram of a finally prepared nanoparticle according to an exemplary embodiment of the present invention. For example, as the drug, curcumin (cur) was selected, which exhibits absorption and fluorescence in the visible region by itself, and as the fluorescent dye, 2,2′-(2-((9-ethyl-9H-carbazol-3-yl)methyl ene)-1H-indene-1,3(2H)-diylidene)dimalononitrile (CbV10) was selected, which is a near-infrared fluorescent dye capable of generating fluorescence by using fluorescence from the curcumin as excitation light by fluorescence energy transfer.

FIG. 2 shows the results identifying the shapes of the nanoparticles containing the hydrophobic drug and/or fluorescent dye according to an exemplary embodiment of the present invention by transmission electron microscopy images. Nanoparticles loading only curcumin, which is the drug (NP-cur), nanoparticles loading only CbV10, which is the near-infrared fluorescent dye (NP-CbV10), and nanoparticles simultaneously loading both curcumin and CbV10 (NP-cur/CbV10) were used, respectively, and scale bars are 100 nm.

FIG. 3 shows (a) absorption spectra and (b) fluorescence spectra in the visible region of the nanoparticles (NP-cur, NP-CbV10, and NP-cur/CbV10) containing the hydrophobic drug and/or fluorescent dye according to an exemplary embodiment of the present invention.

FIG. 4 shows fluorescence images of the normal mouse model to which the nanoparticles (NP-cur or NP-CbV10) containing the hydrophobic drug or fluorescent dye according to an exemplary embodiment of the present invention were intravenously administered, and of organs (liver, lung, pancreas, kidney, heart, and brain) removed therefrom, respectively.

FIG. 5 shows fluorescence images of the normal mouse model to which the nanoparticles containing an indane derivative, 2,2′-(1H-indene-1,3(2H)-diylidene)dimalononitrile, as the near-infrared fluorescent dye according to an exemplary embodiment of the present invention were intravenously administered.

FIG. 6 shows fluorescence images over time of the normal mouse model to which a Cy5.5-bound F-127 solution was intravenously injected, and of the organs removed therefrom.

FIG. 7 shows fluorescence images of the normal mouse model to which the nanoparticles (NP-cur/CbV10) containing the hydrophobic drug and fluorescent dye according to an exemplary embodiment of the present invention were intravenously administered, and of the organs removed therefrom. In FIG. 7a, fluorescence (left) generated by irradiating light at a wavelength of 640 nm, which directly excited CbV10 in the nanoparticles, and fluorescence (right) generated from CbV10 by fluorescence energy transfer from irradiating light at a wavelength of 500 nm, which excited curcumin, were measured, respectively. FIG. 7b is a graph showing the intensity of the fluorescence signals according to elapsed time after administration and the wavelengths of the excitation light.

FIG. 8 shows fluorescence images of curcumin over time in normal mice to which the nanoparticles (NP-cur/CbV10) containing the hydrophobic drug and fluorescent dye according to an exemplary embodiment of the present invention were intravenously administered.

FIG. 9 shows bioluminescence (BL) and photoluminescence (PL) images of the luciferase-expressing brain tumor model measured after intravenously injecting luciferin and NP-CbV10 according to the present invention.

FIG. 10 shows optical and fluorescence images of brain tissue sections removed from the brain tumor model to which the nanoparticles (NP-cur/CbV10) containing the hydrophobic drug and fluorescent dye according to an exemplary embodiment of the present invention were intravenously administered. The red arrow indicates the location of the cancer tissue.

FIG. 11 shows changes in bioluminescence depending on NP-cur administration periods in the bioluminescent glioblastoma multiforme (GBM) model to which NP-cur according to the present invention was intravenously injected. FIG. 11a shows bioluminescence images obtained from the GBM brain tumor model, and FIG. 11b is a graph showing the intensity of the signals obtained therefrom by digitization.

FIG. 12 shows examples of 1,3-indandione-based derivatives comprising a carbazole ring, as near-infrared fluorescent substances.

FIG. 13 shows examples of 1,3-indandione-based derivatives comprising an aniline ring, as near-infrared fluorescent substances.

FIG. 14 shows examples of a series of indane derivatives as near-infrared fluorescent substances.

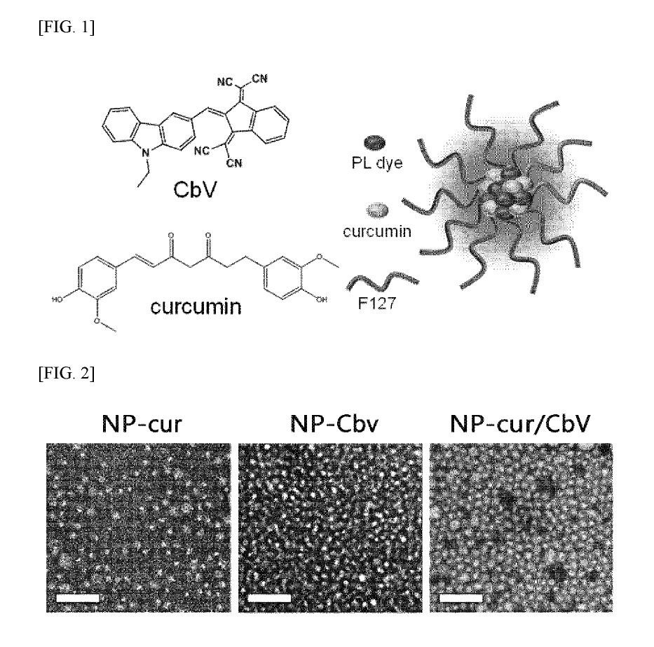

### DETAILED DESCRIPTION OF THE EMBODIMENTS

Hereinbelow, the present invention will be described in detail with accompanying exemplary embodiments. However, the exemplary embodiments disclosed herein are only for describing the invention more specifically and should not be construed as limiting the scope of the present invention.

### EXAMPLE 1

### Preparation of Nanoparticles Containing Hydrophobic Drug and/or Fluorescent Dye

The nanoparticles containing the drug and/or fluorescent dye according to the present invention were prepared by, for example, dispersing in water curcumin, which is an anticancer agent, as the drug, and/or a carbazole derivative of an arylvinyl compound which is a fluorescent dye absorbing near-infrared ray, with an amphiphilic polymer as a homogeneous mixture, thereby inducing to spontaneously and uniformly form a colloid. No precipitates were observed in such dispersion, and it indicates that the drug and/or fluorescent dye molecules, which are water-insoluble, were successfully loaded in the hydrophobic interior space of the self-assembled nanostructure of the amphiphilic polymer. The composition of such particles and a specific preparation method thereof are as follows.

### 1-1. Preparation of Curcumin-Encapsulated Amphiphilic Polymeric Nanoparticles (NP-cur)

In order to prepare curcumin-encapsulated amphiphilic polymeric nanoparticles (NP-cur), 0.1 mg of curcumin (Sigma-Aldrich) and 20 mg of Pluronic F-127, which is an amphiphilic polymer, were completely dissolved in 0.5 mL of dichloromethane (Junsei), followed by airblowing to selectively evaporate only the solvent. 2 mL of water was added to the dried mixture, and it was dispersed uniformly with an ultrasonic disperser. The concentration of curcumin contained in the finally prepared curcumin-encapsulated amphiphilic polymeric nanoparticle dispersion was 50 μg/mL.

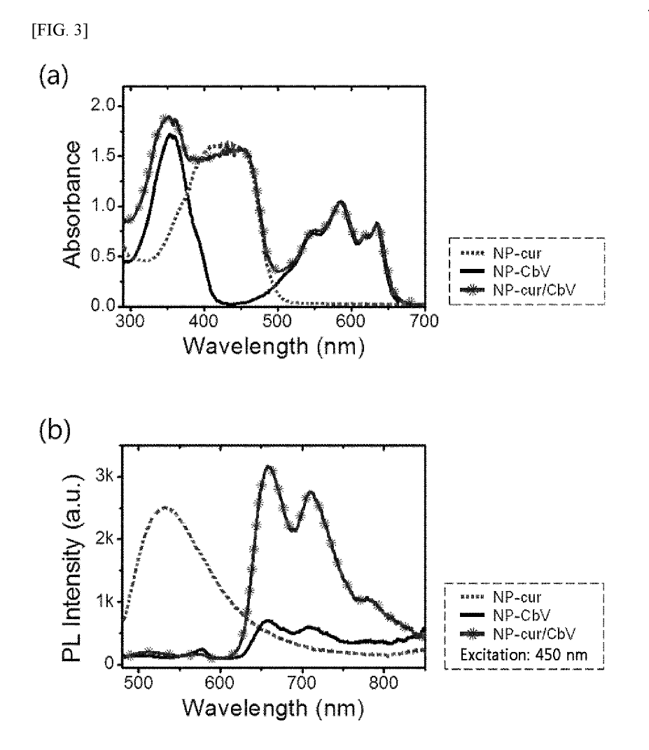

Transmission electron microscopy (TEM) images of the NP-cur prepared as described above were measured, and the result is shown in FIG. 2 (see left side of FIG. 2). The average diameter of the NP-cur particles calculated from the TEM images of FIG. 2 was 11±2.5 nm. In order to confirm whether curcumin was loaded inside the prepared nanoparticles, the absorption/emission spectra of curcumin were measured. As a result, it was confirmed that due to the loading of curcumin, absorption at a maximum absorption wavelength of 432 nm and fluorescence at a maximum emission wavelength of 532 nm appeared in the ultraviolet-visible region (FIG. 3).

### 1-2. Preparation of Amphiphilic Polymeric Nanoparticles (NP-CbVn or NP-AnVm) Containing Near-Infrared Dluorescent Dye 1

Except that 0.2 mg of a series of 1,3-indandione-based derivatives containing a carbazole ring or an aniline ring, for example, 2,2′-(2-((9-ethyl-9H-carbazol-3-yl)methylene)-1H-indene-1,3(2H)-diylidene)dimalononitrile (CbV10), (Z)-2-(2-((9-ethyl-9H-carbazol-3-yl)methylene)-3-oxo-2,3-dihydro-1H-inden-1-ylidene)malononitrile (CbV9), (Z)-2-(2-(4-(dimethylamino)benzylidene)-3-oxo-2,3-dihydro-1H-inden-1-ylidene)malononitrile (AnV9), and 2,2′-(2-(4-(dimethylamino)benzylidene)-1H-indene-1,3(2H)-diylidene)dimalononitrile (AnV10) was used as the near-infrared fluorescent dye instead of curcumin, CbVn or AnVm-encapsulated amphiphilic polymeric nanoparticles (NP-CbVn and NP-AnVm) were prepared in the same manner as in Example 1-1. The concentration of CbVn or AnVm contained in the finally prepared CbVn or AnVm-encapsulated amphiphilic polymeric nanoparticle dispersion was about 100 μg/mL. For reference, CbVn or AnVm was synthesized by referencing the methods disclosed in ACS Appl. Mater. Interfaces, 2013, 5: 8881.

TEM images of the NP-CbV10 prepared as described above were measured, and the result is shown in FIG. 2 (see middle of FIG. 2). The average diameter of the NP-CbV10 particles calculated from the TEM images of FIG. 2 was 10±1.2 nm. The absorption/emission spectra of curcumin were measured in order to confirm whether CbV10 was loaded inside the prepared nanoparticles. As a result, due to the loading of CbV10, it was confirmed that absorption at maximum absorption wavelengths of 353 nm, 586 nm, and 634 nm appeared in the ultraviolet-visible region, and fluorescence at maximum emission wavelengths of 662 nm and 707 nm appeared in the near-infrared region (FIG. 3). In the same manner, the absorption and emission spectra of the prepared nanoparticles were measured to confirm whether CbV9, AnV9, and AnV10 were loaded inside the nanoparticles. As a result, it was confirmed that the nanoparticles loading CbV9 had a maximum absorption wavelength of 550 nm and a maximum emission wavelength of 732 nm, the nanoparticles loading AnV9 had a maximum absorption wavelength of 552 nm and a maximum emission wavelength of 656 nm, and finally, the nanoparticles loading AnV10 had maximum absorption wavelengths of 584 nm and 634 nm and maximum emission wavelengths of 658 nm and 710 nm.

### 1-3. Preparation of Amphiphilic Polymeric Nanoparticles Containing Near-Infrared Fluorescent Dye 2

Except that a series of indane derivatives were used as the near-infrared fluorescent dye instead of a series of 1,3-indandione derivatives containing a carbazole ring or an aniline ring, the nanoparticles loading the near-infrared fluorescent dye were prepared in the same manner as in Example 1-2. As the indane derivatives, a total of 9 types of commercially available compounds were purchased from the company TCI, etc. and used, and the types are as follows:
2,2′ -(1H-indene-1,3(2H)-diylidene)dimalononitrile,
2,2′ -(1H-indene-1,3(2H)-diylidene)dimalonic acid,
(2E,2′Z)-diethyl 2,2′-(1H-indene-1,3(2H)-diylidene)bis(2-cyanoacetate),
(2E,2′Z)-2,2′-(1H-indene-1,3(2H)-diylidene)bis(2-cyanoacetic acid),
(E)-ethyl 2-cyano-2-(3-(dicyanomethylene)-2,3-dihydro-1H-inden-1-ylidene)acetate,
(2,2′,2″-([1,2′-biindenylidene]-1′,3,3′(2H)-triylidene)trimalonic acid,
2,2′,2″-((1Z,1′Z,3E,3′E)-[1,2′-biindenylidene]-1′,3,3′(2H)-triylidene)tris(2-cyanoacetic acid),
triethyl 2,2′,2″-((1Z,1′Z,3E,3′E)-[1,2′-biindenylidene]-1′,3,3′(2H)-triylidene)tris(2-cyanoacetate), and
2,2′,2″-([1,2′-biindenylidene]-1′,3,3′(2H)-triylidene)trimalononitrile.

### 1-4. Preparation of Amphiphilic Polymeric Nanoparticles (NP-cur/CbV) Containing Curcumin and Fluorescent Dye

Except that 0.2 mg of CbV was additionally used in addition to 0.1 mg of curcumin, the amphiphilic polymeric nanoparticles (NP-cur/CbV) containing curcumin and CbV were prepared in the same manner as in Example 1-1. The concentrations of curcumin and CbV contained in the finally prepared amphiphilic polymeric nanoparticle dispersion containing curcumin and CbV were 50 μg/mL and 100 μg/mL, respectively.

TEM images of the NP-cur/CbV as prepared above were measured, and the result is shown in FIG. 2 (see right side of FIG. 2). The average diameter of the NP-cur/CbV particles calculated from the TEM images of FIG. 2 was 14±1.7 nm. In order to confirm whether curcumin and CbV were loaded inside the prepared nanoparticles, the absorption/emission spectra of curcumin were measured. As a result, it was confirmed that due to the simultaneous loading of the above two types of photonic molecules, curcumin and CbV, showed absorption at maximum absorption wavelengths of 432 nm, 586 nm, and 634 nm in the visible region and showed fluorescence at maximum emission wavelengths of 662 nm and 707 nm in the near-infrared region according to fluorescence energy transfer between these molecules (FIG. 3).

As described above, the optical properties of the amphiphilic polymeric nanoparticles loading an optically active drug and/or fluorescent dye molecule prepared according to Examples 1-1 to 1-4 were consistent with the intrinsic optical properties which were displayed when the optically active drug loaded therein was dissolved in an organic solvent. It was shown therefrom that the optically active drug and/or fluorescent dye was successfully loaded in the interior space of the amphiphilic polymeric nanoparticle, and thus it was confirmed that even when concentrated at a high density in the interior space of the nanoparticle, it exhibited effective fluorescence without self-quenching by adjacent molecules.

Meanwhile, it was confirmed that, when irradiating excitation light at a wavelength of 450 nm, at which the absorption of curcumin is high and the absorption by CbV is hardly displayed, to NP-cur/CbV simultaneously loading both curcumin and CbV, the fluorescence of curcumin due to the 450 nm excitation light was quenched and the fluorescence of CbV increased at the same time. It can be explained by the fluorescence energy transfer which occurs because the fluorescence of curcumin occurring under excitation at 450 nm is spectrally overlapped with the absorption wavelength range of CbV, and such molecules are highly densely packed inside the nanoparticles thereby placing within a distance of several nanometers. Therefore, in subsequent experiments, drug release from the particles was non-invasively confirmed using the fluorescence energy transfer phenomenon between curcumin and CbV.

### EXAMPLE 2

### Brain Accumulation and Drug Release Behavior Following the Crossing of the Blood-Brain Barrier after Intravenous Injection of Drug-Containing Amphiphilic Polymeric Nanoparticles

### 2-1. Evaluation of Brain Accumulation Property after Intravenous Injection of Drug-Containing Nanoparticles in Normal Mouse Model

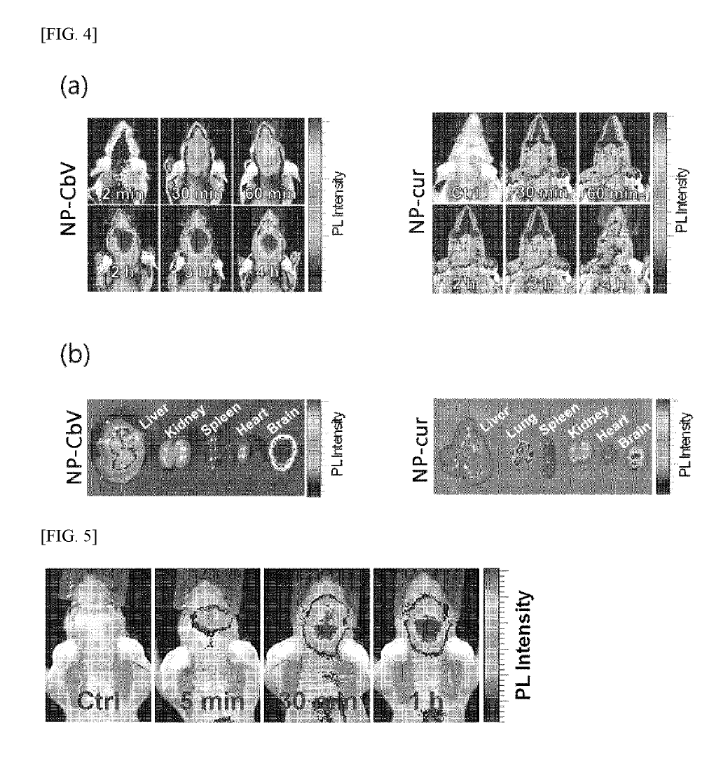

200 μL of the NP-cur or NP-CbV aqueous dispersion prepared according to Examples 1-1 and 1-2 was injected in tail veins of male mice (CAnN.Cg-Foxnlnu/Crl, 5 weeks old, Orient Bio, Korea). At certain time intervals before the intravenous injection and thereafter for four hours, fluorescence signals were tracked and observed using a fluorescence imaging device (IVIS-Spectrum; Perkin-Elmer, USA), and the results are shown in FIG. 4a. Further, immediately after measuring fluorescence images in vivo, the mice were dissected, major organs including the brain were removed, the fluorescence signals generated therefrom were observed, and the results are shown in FIG. 4b.

Further, as described above, after intravenous injection in mice using the aqueous dispersion of the nanoparticles loading 2,2′-(1H-indene-1,3(2H)-diylidene)dimalononitrile prepared according to Example 1-3, the fluorescence signals were tracked and observed using the fluorescence imaging device, and the results are shown in FIG. 5.

### 2-2. Drug Release Property from Nanoparticles Accumulated in Brain after Intravenous Injection in Normal Mouse Model

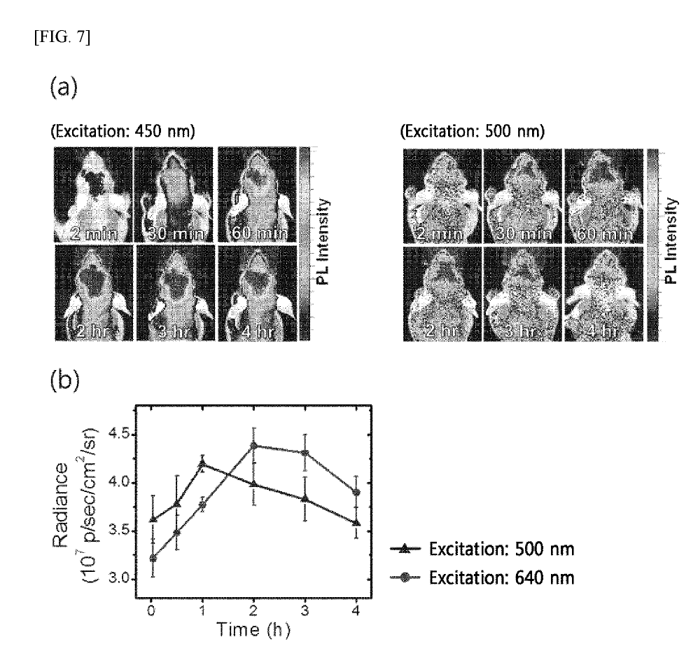

200 μL of the NP-cur/CbV aqueous dispersion prepared according to Example 1-4 was injected in the tail veins of male mice. At certain time intervals before the intravenous injection and thereafter for four hours, fluorescence signals were tracked and observed using the fluorescence imaging device, and the results are shown in FIG. 7. Specifically, fluorescence images were obtained by irradiating light having a wavelength of 640 nm, which can directly excite CbV molecules, and by irradiating light having a wavelength of 500 nm, which can indirectly excite CbV molecules through the fluorescence energy transfer from curcumin, these are shown in FIG. 7a, and changes in fluorescence intensity detected in the brain over time for each excitation wavelength are shown in FIG. 7a.

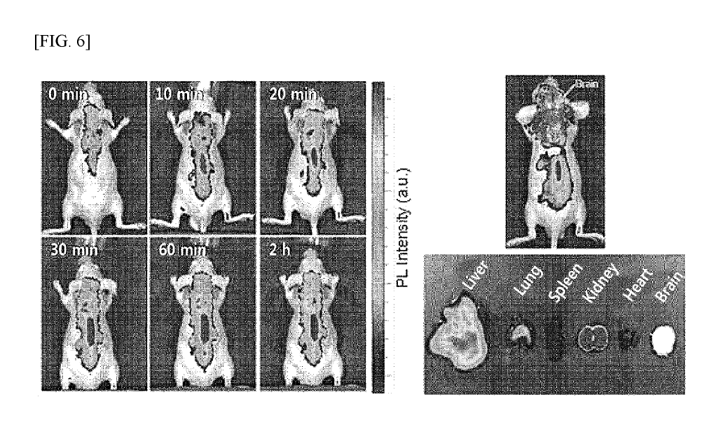

As described above, through Examples 2-1 and 2-2, the nanoparticles loading the drug and/or fluorescent dye according to the present invention were systemically administered to the normal mouse model via intravenous injection, and the behavior of the particles in vivo was monitored by fluorescence imaging to determine whether they could cross the blood-brain barrier (BBB). As shown in FIGS. 4a and 5, as a result of monitoring the whole body with fluorescence imaging after administering the nanoparticles each containing curcumin, or an indane derivative or CbV as a near-infrared fluorescent dye, all of these showed strong fluorescence signals in the brain. It indicates that the nanoparticles according to the present invention, regardless of the type of drug contained therein, were accumulated in the brain by crossing the blood-brain barrier upon systemic administration. Furthermore, as shown in FIG. 4b, fluorescence images of each organ removed by sacrificing after 4 hours of systemic administration of the nanoparticles showed the same pattern and exhibited the strongest fluorescence in the brain. This is a phenomenon different from a that in which usual drug delivery systems are filtered by the reticuloendothelial system (RES) of the liver and spleen and accumulated therein. The nanoparticles of the present invention were designed so that filtration by RES was remarkably reduced due to the size of nanometer-scale ultrafine particles and the surface properties of the nanoparticles formed by self-assembly of an amphiphilic polymer and they can cross the BBB, and they are considered to be able to circulate in the blood for a long time and be accumulated in the brain. This can be supported by the fact that when the Pluronic F-127 solution to which Cy5.5 was bound as a near-infrared fluorescence substance, which cannot form nanoparticles, was intravenously injected, it was not accumulated in the brain (FIG. 6). In conclusion, it is shown that nanostructure formation of amphiphilic polymers is required for drug delivery to the brain by effectively crossing the BBB.

However, the fluorescence signals in the brain after administration of NP-cur or NP-CbV showed a tendency to increase over time and then decrease again, the increase in the fluorescence signals in the brain over time was due to the fact that after the particles loading CbV or curcumin were systemically administered, they were circulating through the blood and were accumulated in the brain, and the decrease in the signals was due to the release and dispersion of CbV or curcumin molecules from the nanoparticles delivered to the brain.

In order to confirm the release of curcumin from the NP-cur delivered to the brain, according to Example 2-2, using the nanoparticles (NP-cur/CbV) simultaneously loading both curcumin and CbV, which is a fluorescent dye capable of fluorescence energy transfer therewith, the energy transfer property between these optically active molecules was observed. As shown in FIG. 7a, similar to the nanoparticles loading either curcumin or CbV, it was confirmed that the nanoparticles loading both of these molecules were accumulated in the brain upon intravenous injection. In particular, fluorescence images obtained by irradiating excitation light at 640 nm, which is the absorption wavelength of CbV, clearly showed an increase in the fluorescence signals in the brain over time, similar to the result for the nanoparticles loading only CbV (up to 2 to 3 hours). Meanwhile, fluorescence images of CbV obtained by irradiating excitation light at 500 nm, which is the absorption wavelength of curcumin, showed a tendency in which fluorescence signals over time were increased up to 60 minutes and then rapidly decreased thereafter. This was due to the fact that the CbV molecules were still present inside the nanoparticles while the curcumin molecules were released out of the particles, and the distance between these molecules increased so that the fluorescence energy transfer between these molecules was no longer possible. The changes in the fluorescence signals according to the wavelengths of the excitation light, which are shown in FIG. 7b by comparison, indicated that initially, the fluorescence signals generated from CbV through the fluorescence energy transfer therefrom by exciting curcumin, which acted as a fluorescence donor, were stronger, but as time passed, fluorescence through the fluorescence energy transfer was rapidly reduced, and the fluorescence signals generated by directly exciting CbV became stronger. However, in terms of fluorescence of CbV, although higher fluorescence intensity due to CbV was maintained for a longer period of time in the case of the nanoparticles loading only CbV, meanwhile in the case of the nanoparticles simultaneously loading both curcumin and CbV, the fluorescence intensity of CbV itself gradually began to decrease after the passage of 2 hours, and after 3 hours, a more remarkable decrease in the fluorescence signals was observed, which was considered to be because of the gradual release of CbV due to the destruction of the nanoparticle structure caused by the release of the drug loaded together or because of the release of the nanoparticles themselves in the brain. On the other hand, the fluorescence of curcumin itself showed a tendency to increase continuously in the brain in the same time range (FIG. 8). This indicates that the fluorescence of curcumin itself was quenched due to the fluorescence energy transfer to CbV, which is a fluorescent receptor, loaded together, and upon the release thereof from the nanoparticles, the distance thereof to CbV was increased so that the fluorescence energy transfer was reduced, and the fluorescence of curcumin itself was restored.

### EXAMPLE 3

### Lesion-specific Accumulation and Tumor Growth Suppression Property of Nanoparticles in Brain Tumor Model

In order to non-invasively confirm the accumulation specific to the brain, particularly to tumor lesions of the nanoparticles loading the drug according to the present invention and the tumor treating effect due to the release of the drug to the lesions according to the same, a bioluminescent glioblastoma multiforme model was used. Since the bioluminescent glioblastoma multiforme model exhibits bioluminescence in brain tumor lesions upon administration of luciferin, the position and/or size of formed lesions can be confirmed therefrom. Therefore, it became possible to monitor whether the lesion was targeted by comparison of the position of a lesion confirmed by the bioluminescence and the position at which the nanoparticles of the present invention were accumulated, which was obtained through fluorescence images.

### 3-1. Lesion-Specific Accumulation of Nanoparticles in Brain Tumor Model

In order to identify the accumulation behavior of the drug-containing nanoparticles of the present invention to cancerous lesions after crossing the blood-brain barrier, bioluminescence and fluorescence signals were observed using the bioluminescent glioblastoma multiforme (GBM) model. After drilling a fine hole 3.5 mm deep with a 26G needle (Hamilton Company, USA) at a point 0.2 mm posterior to and 2.2 mm lateral from the cranial bregma of a male mouse (CAnN.Cg-Foxnlnu/Crl, 5 weeks old, Orient Bio, Korea), 1.5 μL of physiological saline in which 1×10⁵ luciferase-expressing U-87 MG cells were stably dispersed was administered through the hole.

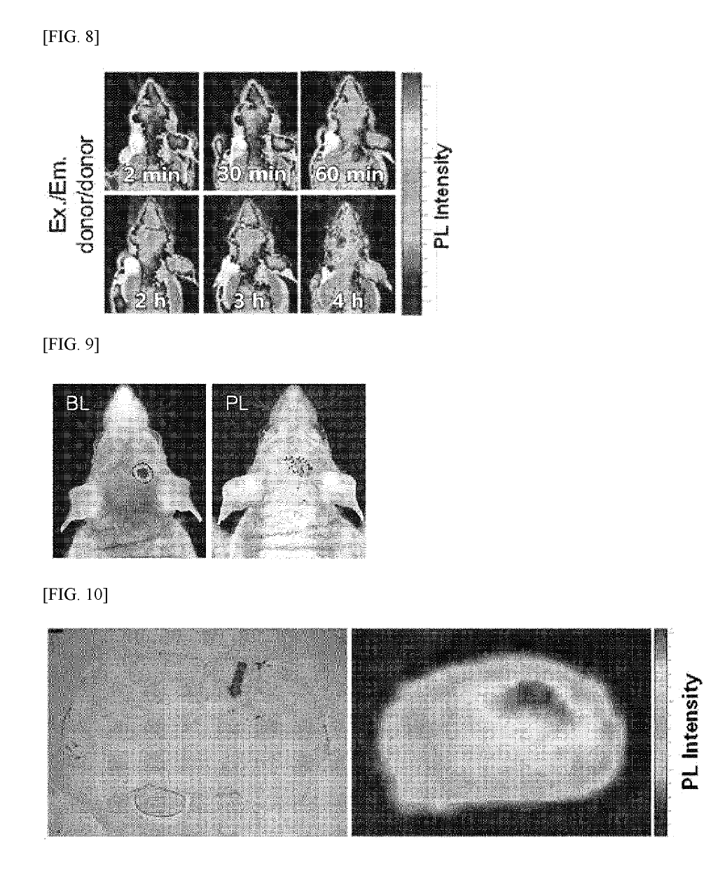

7 days after transplantation of cancer cells, luciferin dissolved in physiological saline was intraperitoneally injected at a dosage of 150 mg/kg, then after 20 minutes, optical images were collected to observe bioluminescence signals. Thereafter, 200 μL of the NP-CbV aqueous dispersion prepared according to Example 1-2 was intravenously injected into the same mouse, and optical images were collected at certain time intervals to observe near-infrared fluorescence signals. The bioluminescence signals and near-infrared fluorescence signals collected therefrom were compared and analyzed, and are shown in FIG. 9. As shown in FIG. 9, the bioluminescence signals were concentrated in specific areas of the brain, and the fluorescence signals from NP-CbV were also concentrated at similar locations. This indicates that NP-CbV according to the present invention was intensively accumulated in the brain tumor lesions which were confirmed by the bioluminescence signals, that is, NP-CbV selectively targeted the brain tumor lesions. Furthermore, brain tissues were removed to observe bioluminescence and fluorescence images which were consistent with the in vivo results (FIG. 10).

### 3-2. Cancer Treating Effect by Administration of Drug-Containing Nanoparticles in Brain Tumor Model

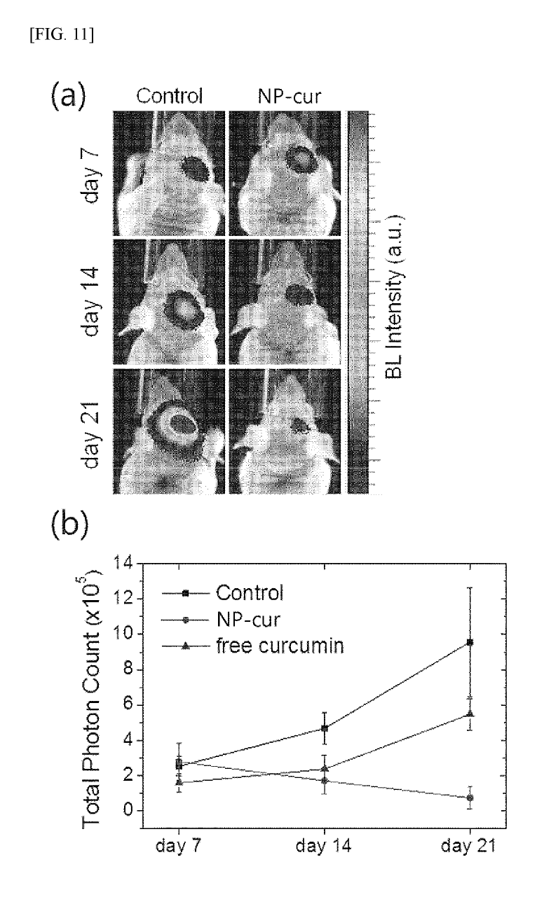

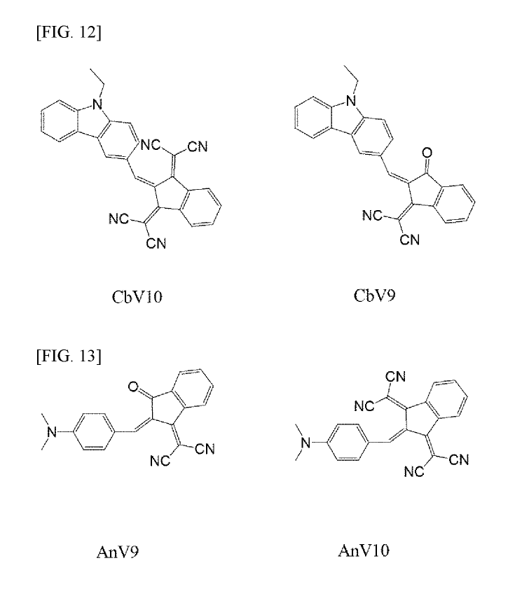

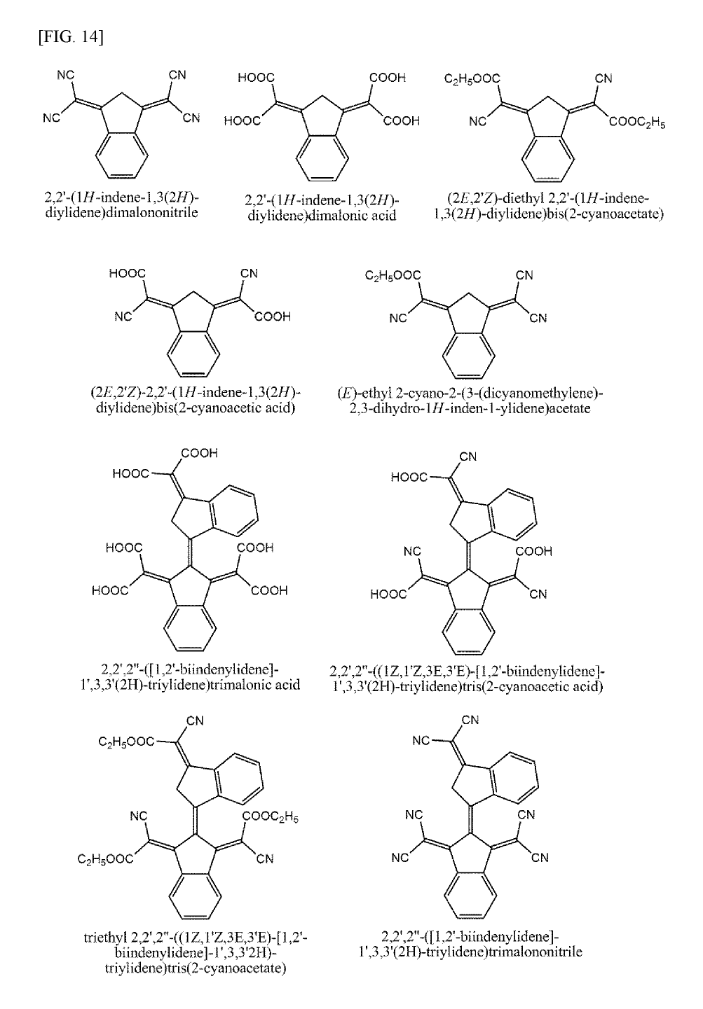

The NP-cur aqueous dispersion prepared according to Example 1-1 was intravenously injected daily at a dose of 200 μL from day 3 of the transplantation of the cancer cells in the brain tumor model prepared according to Example 3-1. Luciferin was administered on days 7, 14, and 21 after the administration of the NP-cur aqueous dispersion was initiated, and bioluminescence signals were observed in the same manner as in Example 3-1. For the control groups to which the drug was not administered, a group (control of FIG. 11) to which the F-127 aqueous solution was intravenously injected daily for 3 weeks at a dose of 200 μL, and a group (free curcumin of FIG. 11) to which curcumin which was not loaded in the polymeric nanoparticles but was dissolved in an organic solvent, dimethyl sulfoxide (DMSO, Daejung Chemicals & Metals Co., LTD., Korea), was intraperitoneally injected at a dosage of 360 mg/kg daily for 3 weeks was selected to observe the bioluminescence signals in the same manner as in the NP-cur administration experimental group. Finally, the bioluminescence signals observed in the two control groups and experimental group and the results of comparative analysis thereof are shown in FIG. 11.

As shown in FIG. 11a, as confirmed by bioluminescence, in the control group, that is, the group to which only the F-127 solution was administered, the intensity of luminescence as well as the area thereof was increased, but in the experimental group to which the nanoparticles loading curcumin were administered, the intensity of luminescence and the area thereof were remarkably decreased. This was considered to be because in the control group, due to the absence of the drug, tumor development was not suppressed at all so that the size of the lesion was rapidly increased, whereas in the experimental group, tumor growth was suppressed due to selective and efficient drug delivery to the brain tumor lesions by NP-cur administration, and the size of the lesion was rather remarkably decreased. Furthermore, as shown in FIG. 11b shown in a graph following digitization thereof, the bioluminescence intensity for the experimental group administered with NP-cur was decreased over time, but it was steadily increased in the two control groups. When curcumin, which is a known anticancer agent, was injected in a solution phase, although the rate of increase thereof was decreased, it still showed a large signal increase. In particular, as compared with curcumin (360 mg/kg/d) administered in a solution state, NP-cur according to the present invention was administered (0.2 mg/kg/d) at a dosage as low as 1/1,800 based on the amount of curcumin, and in the case of administration of curcumin in the solution state, even when administrated at a relatively high dose of about 2,000 times thereof, the tumor growth was only slightly slowed, but when administered in the form of the nanoparticles according to the present invention, it was confirmed that it was possible to effectively suppress the growth of the tumor and to further treat the tumor by administering only a significantly reduced amount. This means that when the drug is administered in the form of the nanoparticles loading the drug according to the present invention, it is possible to show a therapeutic effect of reducing the absolute size of tumor, not just at a level of decreasing the growth rate of the tumor. Moreover, since it is used at a remarkably low dosage, this indicates that it is possible to minimize undesirable effects which may occur in normal tissues due to the use of anticancer agents.

1. A drug delivery system (DDS), which crosses the blood-brain barrier (BBB), comprising:
a self-assembled structure having an average diameter of 5 nm to 20 nm of a core-shell structure with a hydrophobic core and a hydrophilic shell, in which a hydrophobic block of an amphiphilic block copolymer is positioned inside the structure and a hydrophilic block of the amphiphilic block copolymer is positioned out of the structure, respectively, and the amphiphilic block copolymer comprises the hydrophilic block of poly(ethylene oxide) (PEO) and the hydrophobic block of poly(propylene oxide) (PPO); and
a hydrophobic drug supported in the hydrophobic core of the self-assembled structure, wherein the ratio of the amphiphilic block copolymer to the hydrophobic drug is adjusted for crossing the blood-brain barrier.

2. The drug delivery system of claim 1, wherein the drug delivery system comprises the hydrophobic drug and the amphiphilic block copolymer at a mass ratio (w/w) of (0.025 to 2):20.

3. The drug delivery system of claim 1, wherein the amphiphilic block copolymer is a triblock copolymer having an average molecular weight of 1,500 Da to 20,000 Da.

4. The drug delivery system of claim 1, wherein the hydrophobic drug is an anticancer agent, antioxidant agent, anti-inflammatory agent, contrast agent, or combination thereof.

5. The drug delivery system of claim 1, wherein the drug delivery system further comprises a near-infrared fluorescent substance in the hydrophobic core of the self-assembled structure.

6. The drug delivery system of claim 5, comprising the near-infrared fluorescent substance at a mass ratio (w/w) of (0.025 to 0.5):20 relative to the amphiphilic block copolymer, wherein the sum of the masses of the hydrophobic drug and near-infrared fluorescent substance is at a mass ratio of (0.05 to 2.5):20 relative to the mass of the amphiphilic block copolymer.

7. The drug delivery system of claim 5, wherein the near-infrared fluorescent substance is an indane derivative or a 1,3-indandione-based derivative comprising a carbazole ring or aniline ring exhibiting fluorescence in a range of 640 nm to 1,000 nm.

8. A pharmaceutical composition for preventing or treating cerebral diseases, comprising the drug delivery system of claim 1 as an active ingredient.

9. The composition of claim 8, wherein the pharmaceutical composition is provided in an injectable form.

10. A method for preparing a drug delivery system, which crosses the blood-brain barrier, having an average diameter of 5 nm to 20 nm of a core-shell structure with a hydrophilic shell, a hydrophobic core and a hydrophobic drug supported therein, comprising:
a first step of airblowing a solution containing a hydrophobic drug, and a amphiphilic block copolymer comprising a hydrophilic block of poly(ethylene oxide) (PEO) and a hydrophobic block of poly(propylene oxide) (PPO) in an organic solvent, in which the ratio of the amphiphilic block copolymer to the hydrophobic drug is adjusted for crossing the blood-brain barrier, to evaporate the organic solvent; and
a second step of adding a water-containing solvent to the product obtained from the previous step followed by dispersion by an ultrasonic disperser.

11. The method of claim 10, wherein the organic solvent is dichloromethane, tetrahydrofuran, chloroform, or a mixture thereof.

12. The method of claim 10, wherein the water-containing solvent of the second step is added to a final concentration in a range of 5 mg/mL to 50 mg/mL based on the amount of the amphiphilic block copolymer used in the first step.

13. The method of claim 10, wherein the solution of the first step further comprises a near-infrared fluorescent substance.

14. The method of claim 13, wherein the near-infrared fluorescent substance is selected to have an absorption spectrum in the fluorescence wavelength range of the hydrophobic drug.
